(19) United States
(12) Patent Application Publication
(10) Pub. No.: US 2024/0367170 A1
(43) Pub. Date: Nov. 7, 2024

(54) REAGENT PRE-LOADING SYSTEM AND MEASURING DEVICE

(71) Applicant: Haemograph Pty Ltd, Bayswater, Victoria (AU)

(72) Inventors: Alexander Samuel LUBANSKY, Bayswater (AU); Martina DI VENERE, Bayswater (AU)

(73) Assignee: Haemograph Pty Ltd, Bayswater, Victoria (AU)

(21) Appl. No.: 18/689,343

(22) PCT Filed: Sep. 6, 2022
(86) PCT No.: PCT/AU2022/051081

§ 371 Date: Mar. 5, 2024

(30) Foreign Application Priority Data
Sep. 6, 2021 (AU) 2021902880

(51) Int. Cl. B01L 3/00

(52) U.S. Cl. CPC B01L 3/50273; B01L 3/52; B01L 2200/04; B01L 2200/16; B01L 2300/0663; B01L 2400/0622

(57) ABSTRACT

A reagent pre-loading system for a measuring device includes: a reagent admission arrangement arranged for receiving a reagent into the reagent pre-loading system; a sample admission arrangement arranged for receiving a sample fluid to be measured into the reagent pre-loading system, wherein the reagent admission arrangement and the sample admission arrangement are each arranged in selective fluid communication with a sample and reagent combination conduit for combining the sample fluid and the reagent; wherein the sample admission arrangement and the reagent admission arrangement are arranged to selectively: whilst the sample and reagent combination conduit is closed to fluid communication with the sample fluid at the sample admission arrangement, receive the reagent into the sample and reagent combination conduit so as to expel air from the sample and reagent combination conduit as the reagent is received therein and prime the sample and reagent combination conduit with the reagent.

### TECHNICAL FIELD

The present disclosure relates to a reagent pre-loading system for use in a measuring device such as a point of care diagnostic measuring device, in particular for use in a rheometer. It also relates to a method of pre-loading reagent into a reagent pre-loading system.

### BACKGROUND

A microfluidic rheometer is a measuring device that has the function of measuring viscosity, for example of blood samples. It may be desirable to obtain a rapid evaluation of the viscosity of a patient's blood as such information is valuable in assessing the administration of coagulants and anticoagulants to patients. It may also predict the likelihood of a bleed or thrombotic event in those that have a propensity to bleed or are at increased risk of clotting. One aspect of measuring blood viscosity in a rheometer is that the sample blood may be reacted with different reagents (for example, anti-coagulants) across a plurality of parallel channels of a cartridge, where the blood and reagent are mixed before reaching the measuring component of the rheometer. At the initial loading stage it is fundamental that, once a blood sample is taken from the patient, it is rapidly introduced into the cartridge to avoid unwanted and uncontrolled coagulation. If such coagulation occurs, the rheological properties of the blood change and the measurement output can be misleading. Therefore, there should ideally be as few barriers as possible between the blood sample being removed from the patient and measurement commencing.

One such barrier before measurement involves mixing the blood sample with appropriate reagents. Reagents could be mixed with the blood sample externally of the measurement device, but this requires a time-consuming step. Alternatively, the reagents can be loaded into the measurement device alongside the blood sample, but this requires precise control and increases the risk of uncontrolled and/or inhomogeneous mixing. A third option is for the reagents to be pre-loaded into the cartridge before the blood sample is added to the cartridge.

By pre-loading the reagent into the cartridge, the measurement device will be setup and ready to use when blood is taken from the patient at the point of care. However, when two fluids are introduced at different times into channels of a fluidic device, the air present in the channels cannot be easily removed. As a consequence, the air forms an air pocket or gap between the two fluids and prevents good mixing between the two fluids. The presence of air in the mixture will adversely affect the accuracy of measurement of the mixture properties. Where the two fluids concerned are blood and the reagent, and the property to be measured is the viscosity of the mixture of the blood and the reagent, measurement accuracy is critical to good patient outcomes. It is therefore crucial that air pockets in the fluid mixture are prevented or removed prior to measurement.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

In a first aspect, a reagent pre-loading system for a measuring device comprises: a reagent admission arrangement arranged for receiving a reagent into the reagent pre-loading system; a sample admission arrangement arranged for receiving a sample fluid to be measured into the reagent pre-loading system, wherein the reagent admission arrangement and the sample admission arrangement are each arranged in selective fluid communication with a sample and reagent combination conduit for combining the sample fluid and the reagent; wherein the sample admission arrangement and the reagent admission arrangement are arranged to selectively: whilst the sample and reagent combination conduit is closed to fluid communication with the sample fluid at the sample admission arrangement, receive the reagent into the sample and reagent combination conduit so as to expel air from the sample and reagent combination conduit as the reagent is received therein and prime the sample and reagent combination conduit with the reagent; whilst the sample and reagent combination conduit is closed to fluid communication with the sample fluid at the sample admission arrangement, receive the sample fluid into the sample admission arrangement so as to expel air from the sample admission arrangement and prime the sample admission arrangement with the sample fluid; and, whilst the sample and reagent combination conduit is open to fluid communication with the sample fluid at the sample admission arrangement, admit further sample fluid into the sample admission arrangement and into the sample and reagent combination conduit for combining with the reagent.

At least one of the reagent admission arrangement and the sample valve arrangement may include an air vent or valve.

The reagent admission arrangement may comprise a reagent valve arrangement configured for selective admission of the reagent into the reagent pre-loading system; and the sample admission arrangement may comprise a sample valve arrangement configured for selective admission of the sample fluid to be measured into the reagent pre-loading system.

The reagent pre-loading system allows the user to pre-load the reagent into the measuring device whilst removing the air present in the measuring device that prevents good mixing. The reagent can be pre-loaded into the measuring device at any point prior to introduction of the sample fluid into the sample and reagent combination conduit, so that the two do not need to be loaded simultaneously and such that they can be loaded with precision of sample volume that is achieved passively without the need for precise user control. The reagent can be pre-loaded close to the point of use of the measuring device so that the shelf-life of the measuring device is not dictated by the storage requirements of the reagents and also to allow selection of reagents based on particular user requirements or patient needs. This is particularly important where the sample fluid is blood and the measurement to be taken is the viscosity of the blood/reagent sample to test the effectiveness of different reagents on the blood viscosity. Different patients will require different reagents to achieve a desired coagulation behaviour and a patient may require different reagents at different times as the effectiveness of a particular reagent on the patient's blood changes over time. It is therefore advantageous to be able to select a desired reagent tailored for a particular use or patient. This requirement precludes pre-loading of the reagent at the time of manufacture of the device. Whilst such a solution may help to resolve the air pocket, it unduly limits the flexibility of the measuring device.

Furthermore, different reagents can have very different storage requirements, for example a first reagent may need to be stored at a temperature of around 4° C., whilst a second reagent may require storage at −70° C. Once removed from those conditions, the reagent may need to be used within a short time frame. These storage requirements are incompatible with one another and do not easily allow for different reagents to be pre-loaded into the measuring device at an earlier stage, for example during manufacture of the device. This flexibility of use of the measuring device may be even more important where more than one reagent pre-loading system is included in the measuring device for the measurement of multiple fluid samples with different reagents. Accordingly, the ability to select a particular reagent or multiple reagents for simultaneous testing and to pre-load the reagent or reagents immediately prior to the use of the measuring device whilst achieving good mixing of the reagent with the blood sample is an important advantage of the reagent pre-loading system.

The reagent valve arrangement may be configured for selective fluid communication between at least a reagent delivery port and the sample and reagent combination conduit. The reagent valve arrangement may also be configured for selective fluid communication between the sample and reagent combination conduit and a mixer. The sample valve arrangement may further comprise a sample fluid conduit arranged for receiving the sample fluid to be measured into the sample valve arrangement. It may be configured for selective fluid communication between each of the sample fluid conduit, the sample and reagent combination conduit and an air vent or valve.

The reagent pre-loading system may be configured to, upon admitting the reagent into the sample and reagent combination conduit, substantially remove air from the sample and reagent combination conduit. In an embodiment, admitting the reagent into the sample and reagent combination conduit and substantially removing air from the sample and reagent combination conduit may comprise: selectively admitting the reagent at the reagent delivery port into the sample and reagent combination conduit whilst the sample valve arrangement is closed to the sample fluid conduit and open to the air vent or valve, such that admission of the reagent into the sample and reagent combination conduit displaces air in the sample and reagent combination conduit and expels it through the air vent or valve.

The admitting the sample fluid so as to expel air from the sample valve arrangement and prime the sample valve arrangement with the sample fluid may comprise: closing the sample valve arrangement to the sample and reagent combination conduit and opening the sample valve arrangement to the sample fluid conduit so as to selectively admit sample fluid into the sample fluid conduit, such that admission of the sample fluid into the sample fluid conduit displaces air in the sample fluid conduit and the sample valve arrangement and expels it through the air vent or valve.

The admitting further fluid sample into the sample valve arrangement and into the sample and reagent combination conduit for combining with the reagent may comprise, whilst the reagent valve arrangement is open to the mixer conduit and closed to the reagent delivery port: closing the sample valve arrangement to the air vent or valve and opening the sample valve arrangement to the sample and reagent combination conduit, such that further sample fluid delivered into the sample fluid conduit is admitted into the sample and reagent combination conduit for combining with the reagent in the sample and reagent combination conduit.

The sample valve arrangement may comprise a three way valve that is controllable to selectively open and/or close fluid access to and/or from the sample fluid conduit, the sample and reagent combination conduit and an air vent or valve.

The reagent valve arrangement may comprise a three way valve that is controllable to selectively open and/or close fluid access to and/or from the reagent delivery port, the sample valve arrangement via the sample and reagent combination conduit and the mixer. The three-way valves of the sample valve arrangement and/or the reagent valve arrangement may comprise a pressure-induced or electronic valve.

Alternatively, the sample valve arrangement may comprise a two way valve providing selective fluid communication between the sample and reagent combination conduit and the air vent or valve; and a one way valve adapted to permit substantially one way fluid communication from the sample fluid conduit into the sample and reagent combination conduit and into the air vent or valve. In a still further embodiment, the two-way valve may be replaced by a one-way valve, e.g. a check valve, providing selective fluid communication from the sample and reagent conduit to the air vent or valve. The reagent valve arrangement may comprise a T-junction conduit arranged in fluid communication with the sample and reagent combination conduit at an upstream end thereof and in fluid communication with a mixer valve at a downstream end thereof, the mixer valve providing selective fluid communication between the T-junction conduit and the mixer; and a check valve adapted to permit substantially one way fluid flow from the reagent delivery port into the T-junction conduit.

The mixer valve may be selected from one of a pressure-induced valve, mechanically active valve, externally controlled pneumatic valve, burst valve or electronic valve.

In a further alternative arrangement the sample valve arrangement may comprise a check valve adapted to permit substantially one way fluid flow from the sample fluid conduit to the air vent or valve, and a check valve adapted to permit substantially one way fluid flow from the sample and reagent combination conduit to the air vent or valve; and a pressure actuated or electronically controlled valve adapted to permit substantially one way fluid flow from the sample fluid conduit into the sample and reagent combination conduit.

The reagent valve arrangement may comprise a T-junction arranged in fluid communication with the sample and reagent combination conduit at an upstream end thereof and in fluid communication with a pressure-induced or electronic valve at a downstream end thereof, the pressure-induced or electronic valve providing selective fluid communication between the T-junction conduit and the mixer conduit; and a check valve adapted to permit substantially one way fluid flow from the reagent delivery port to the T-junction conduit

It will be appreciated by the skilled person that the reagent valve arrangements and/or the sample valve arrangements described in the preceding paragraphs may be interchangeable with one another without departing from the scope of this disclosure.

In some embodiments, the reagent valve arrangement and the sample valve arrangement may be arranged in selected fluid communication via the sample and reagent combination conduit.

In a further embodiment, the reagent valve arrangement and the sample valve arrangement may form at least part of a four-way valve arrangement configured to selectively admit reagent and sample fluid into the sample and reagent combination conduit. The reagent pre-loading system may further comprise a downstream valve arrangement configured for selective expulsion of air in the sample and reagent combination conduit upon admission of reagent therein. The downstream valve arrangement may be selectively configured to permit combined sample fluid and reagent in the sample and reagent combination conduit to pass into a mixer. In an embodiment, the downstream valve arrangement may be a three-way valve providing selective fluid communication between the sample and reagent combination conduit, an air vent or valve, and the mixer.

The four-way valve may include an electronic valve or a pressure induced, mechanically actuated or hydraulically actuated valve. The downstream valve may include an electronic valve or a pressure induced valve.

The reagent valve arrangement may comprise a check valve for selective admission of reagent at the reagent delivery port and/or the sample valve arrangement comprises a check valve for selective admission of sample fluid at the sample fluid conduit.

The reagent pre-loading system may further comprise a controller configured to selectively operate one or more components of the sample valve arrangement and the reagent valve arrangement.

In some embodiments, the reagent pre-loading system may comprise a reagent delivery port, wherein the reagent admission arrangement comprises a pierceable sealed membrane arranged to sealingly close the reagent delivery port to fluid flow there through until it is pierced, and, upon the sealed membrane being pierced, to receive the reagent in the reagent pre-loading system.

The reagent admission arrangement may comprise a valve configured for selective fluid communication between the sample and reagent combination conduit and a mixer.

The sample admission arrangement may be configured for selective fluid communication between each of a sample fluid conduit, the sample and reagent combination conduit and an air vent or valve.

The reagent pre-loading system may be further configured to, upon receiving the reagent into the sample and reagent combination conduit, remove air from the sample and reagent combination conduit.

The sample admission arrangement may comprise a valve arrangement for selective admission of sample fluid into the reagent pre-loading system and selective fluid communication between the sample fluid conduit and the sample and reagent combination conduit, wherein the receiving the sample fluid so as to expel air from the sample admission arrangement and prime the sample admission arrangement with the sample fluid comprises: closing the sample admission arrangement to the sample and reagent combination conduit and opening the sample admission arrangement to the sample fluid conduit so as to selectively admit sample fluid into the sample fluid conduit, such that admission of the sample fluid into the sample fluid conduit displaces air in the sample admission arrangement through the air vent or valve.

In some embodiments, admitting further sample fluid into the sample admission arrangement and into the sample and reagent combination conduit for combining with the reagent comprises, whilst the reagent admission arrangement is open to the mixer and closed to the reagent delivery port: closing the sample admission arrangement to the air vent or valve and opening the sample admission arrangement to the sample and reagent combination conduit, such that further sample fluid delivered into the sample fluid conduit is admitted into the sample and reagent combination conduit for combining with the reagent in the sample and reagent combination conduit.

According to a second aspect, a cartridge for a measuring device comprises the reagent pre-loading system of the first aspect.

According to a third aspect, a measuring device for measuring a material property comprises a sample loading port, a reagent pre-loading system arranged in selective fluid communication with the sample loading port and a closed-ended measuring system arranged in fluid communication with the reagent pre-loading system, whereby the closed-ended measuring system is configured to contain a controlled volume of the sample fluid and reagent to be measured. The measuring device may further comprise a mixer arranged in fluid communication between the reagent pre-loading system and the measuring system.

The measuring device may further comprise a distribution device arranged in selective fluid communication with the sample loading port and adapted for distributing a sample fluid introduced into the sample loading port to a plurality of distribution channels; and a plurality of the reagent pre-loading systems, each of the plurality of the reagent pre-loading systems being arranged in fluid communication with a respective one of the plurality of distribution channels for receiving a portion of the sample fluid for combining with a reagent.

The distribution device may comprise a distributing body having a sample fluid inlet arranged in fluid communication with a plurality of sample fluid distribution channels.

The measuring device may comprise a plurality of the measuring systems, and wherein each of the plurality of reagent pre-loading systems is arranged in fluid communication leading to a measuring system of the plurality of measuring systems. The plurality of measuring systems may be of the same type or they may be of different types for measuring different properties and/or characteristics of the sample fluid, either alone or combined with a chemical or reagent.

The measuring device may comprise a plurality of the mixers, wherein each of the plurality of reagent pre-loading systems is arranged in fluid communication with a mixer of the plurality of mixers.

In some embodiments, the sample fluid may be blood. The measuring device may be a point of care diagnostic measuring device for measuring one or more properties of the sample fluid either alone or mixed with a chemical or reagent. The measuring system of the measuring device may be configured for measuring a rheology of the sample fluid. Whilst the embodiments disclosed herein are described in terms of measuring the rheology of a blood sample, the measuring device is suitable for use in measuring the rheology of many other fluids. Furthermore, the measuring device may alternatively be configured for measuring other properties of the sample fluid, including turbidity, optical characteristics and other parameters. The reagent pre-loading system may be in accordance with the first aspect disclosed herein.

The measuring system of the measuring device is a closed end system whereby the sample fluid to be measured is fully contained within the measuring system. The closed end or dead-ended system minimises the amount of blood and reagent needed to conduct the measurements, which is of particular significance when taking measurements involving a patient's blood which should be conserved as far as possible. It also increases accuracy of the blood to reagent ratio within the measuring system. This requirement of the measuring system makes it even more important that the blood and reagent be well mixed with no air pockets prior to measurement taking place.

According to a fourth aspect, there is provided a method of operating a reagent pre-loading system of a measuring device, the reagent pre-loading system comprising a reagent admission arrangement configured for selective admission of a reagent into the reagent pre-loading system; a sample admission arrangement configured for selective admission of a sample fluid to be measured into the reagent pre-loading system, wherein the reagent admission arrangement and the sample admission arrangement are each arranged in selective fluid communication with the sample and reagent combination conduit; the method comprising: whilst the sample and reagent combination conduit is closed to fluid communication with sample fluid at the sample admission arrangement, pre-loading a reagent into the sample and reagent combination conduit at any time prior to the admission of the sample fluid into the reagent pre-loading system so as to substantially remove air located in the sample and reagent combination conduit and prime the sample and reagent combination conduit with the reagent; whilst the sample and reagent combination conduit is closed to fluid communication with sample fluid at the sample admission arrangement, admitting a sample fluid into the sample admission arrangement so as to expel air from the sample admission arrangement and prime the sample admission arrangement with the sample fluid; and, whilst the sample and reagent combination conduit is open to fluid communication with sample fluid at the sample admission arrangement, admitting further sample fluid into the sample admission arrangement and into the sample and reagent combination conduit for combining with the reagent.

The air located in the sample and reagent combination conduit may be substantially removed via an air vent or valve.

In some embodiments, the reagent admission arrangement may comprise a reagent valve arrangement, configured for selective admission of the reagent into the reagent pre-loading system, and the sample admission arrangement comprises a sample valve arrangement configured for selective admission of the sample fluid into the reagent pre-loading system.

The reagent valve arrangement may be configured for selective fluid communication between each of a reagent delivery port, the sample and reagent combination conduit and a mixer; and the air vent or valve.

The sample valve arrangement may be configured for selective fluid communication between each of a sample fluid conduit, the sample and reagent combination conduit and an air vent; wherein the admitting the reagent into the sample and reagent combination conduit and substantially removing air from the sample and reagent combination conduit comprises: selectively admitting the reagent at the reagent delivery port into the sample and reagent combination conduit whilst the sample valve arrangement is closed to the sample fluid conduit and open to the air vent, such that admission of the reagent into the sample and reagent combination conduit displaces air in the sample and reagent combination conduit through the air vent or valve.

The admitting the fluid sample so as to expel air from the sample valve arrangement and prime the sample valve arrangement with the sample fluid may comprise: closing the sample valve arrangement to the sample and reagent combination conduit and opening the sample valve arrangement to the sample fluid conduit so as to selectively admit sample fluid into the sample fluid conduit, such that admission of the sample fluid into the sample fluid conduit displaces air in the sample fluid conduit and the sample valve arrangement through the air vent.

Admitting further fluid sample into the sample valve arrangement and into the sample and reagent combination conduit for combining with the reagent may comprise, whilst the reagent valve arrangement is open to the mixer conduit and closed to the reagent delivery port: closing the sample valve arrangement to the air vent and opening the sample valve arrangement to the sample and reagent combination conduit, such that further sample fluid delivered into the sample fluid conduit is admitted into the sample and reagent combination conduit for combining with the reagent in the sample and reagent combination conduit.

The reagent admission arrangement may comprise a reagent delivery port, and a pierceable sealed membrane arranged to sealingly close the reagent delivery port to fluid flow there through until it is pierced, and pre-loading the reagent into the sample and reagent combination conduit may comprise piercing the sealed membrane with an external actuator or device and providing the reagent into the reagent pre-loading system via the pierced membrane.

According to a fifth aspect, there is provided a method of measuring a property of a fluid sample using the measuring device of the third aspect, the method comprising: pre-loading a reagent into the reagent loading system of the measuring device at any time prior to admission of the sample fluid into the measuring device so as to substantially remove air located in the sample and reagent combination conduit; admitting a sample fluid into the measuring device so as to expel air from the sample admission arrangement and prime the sample admission arrangement with the sample fluid; and admitting further sample fluid into the sample admission arrangement and into the sample and reagent combination conduit for combining with the reagent; mixing the sample fluid and the reagent and measuring the property of the sample fluid in the measuring system.

### BRIEF DESCRIPTION OF DRAWINGS

Embodiments are described in further detail below, by way of example, with reference to the accompanying drawings briefly described below:

FIG. 1 is a schematic overview diagram of a measuring device;

FIG. 2 is a schematic diagram of the measuring device of FIG. 1;

FIG. 3 is a schematic diagram of a first embodiment of a reagent pre-loading system;

FIG. 4 is a schematic diagram showing a method of operating the reagent pre-loading system of FIG. 3;

FIG. 5 is a schematic diagram of a method of operating a second embodiment of a reagent pre-loading system;

FIG. 6 is a schematic diagram of a third embodiment of a reagent pre-loading system;

FIG. 7 is a schematic diagram of a fourth embodiment of a reagent pre-loading system;

FIG. 8 is a schematic diagram of a distribution system of the measuring device of FIG. 2 and the distribution of a sample fluid to multiple reagent pre-loading systems;

FIG. 9 is a schematic cross-section of a distribution system of the measuring device of FIG. 2;

FIG. 10 is a schematic diagram of a mixer of the measuring device of FIG. 2; and

FIG. 11 is a schematic diagram of a further embodiment of a reagent pre-loading system.

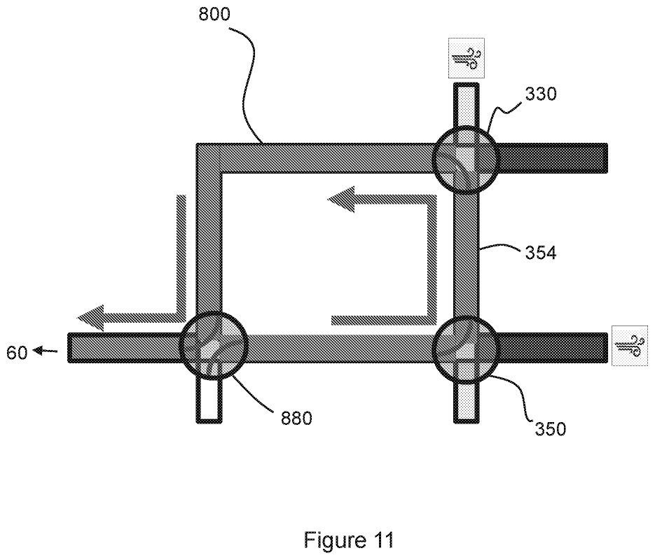

### DESCRIPTION OF EMBODIMENTS

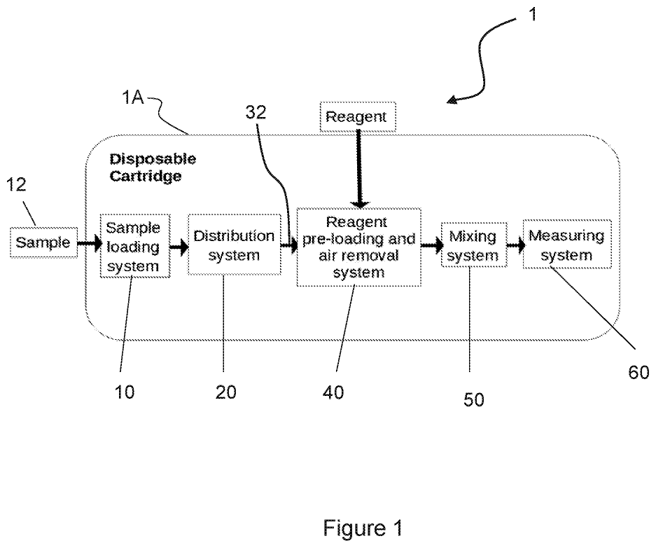

FIG. 1 is a schematic diagram of an overview of a diagnostic measuring device 1 suitable for measuring the viscosity of a fluid. The measuring device 1 comprises a disposable cartridge 1A, for example a microfluidic cartridge, comprising a sample loading system 10 into which a sample of the fluid to be measured may enter the device, a distribution system 20, a reagent pre-loading system 40, mixing system or mixer 50 and a measuring system 60. The cartridge 1A may typically have microfluidic channels having a width dimension of between about 100 um and 1 mm. The cartridge 1A may be made of any suitable material, including but not limited to plastics, especially COC, PMMA and PDMS. In the embodiments described, the sample fluid is blood, however the measuring device 1 is also suitable for use with other fluid materials as will be described herein. Suitable reagents for use with blood include coagulants and anticoagulants. For example, suitable coagulants include calcium, von Willebrand factor, ecarin and arachidonic acid. Suitable anti-coagulants include heparin, citrate, dabigatran and fondaparinux. Other possible reagents include heparinase, idaracizuman, aspirin, and dipyridomole. It will be apparent to the skilled person that the term “reagent” may include one or more of a chemical, a reaction, a reactant, a chemoeffector, an indicator, and a testing agent.

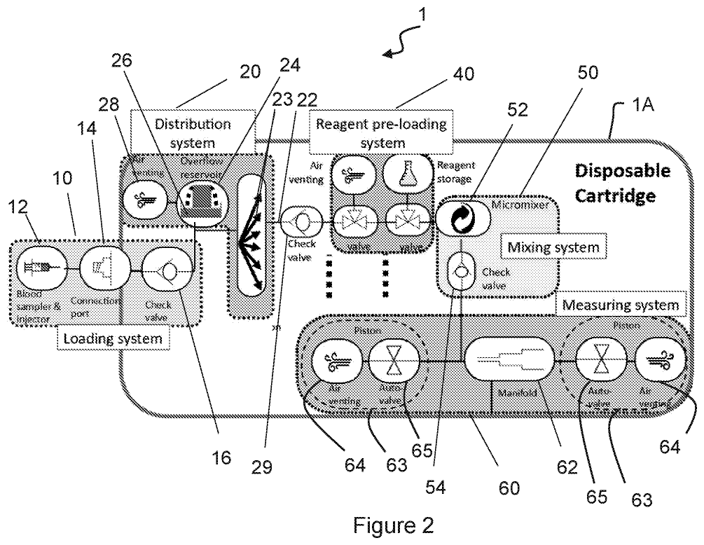

An example embodiment of the measuring device 1 is shown in FIG. 2. The sample loading system 10 is the entry point into the cartridge 1A for the sample material and has the function of introducing blood or other sample material into the cartridge 1 and to prevent any back-flow of the sample material out of the cartridge IA. The sample loading system 10 may be automatic, for example in the form of a sampler, or it may be manual, for example in the form of a syringe 12 as shown in FIG. 2, depending on the user needs. For example, if loading of the sample fluid needs to occur over a limited range of flowrates to achieve good mixing of the sample fluid and the reagent in the mixer 50, an automatic sample loading system may be preferred. However, if sample fluid loading can occur over longer periods of time, e.g. up to 30 s, to achieve an acceptable level of mixing, then a manual sample loading system 10 may be used.

The sample loading system 10 includes a sample fluid port 14 which can be integrated to a check valve 16 to allow the sample material to flow in only one direction, into the cartridge 1A. The sample loading system 10 is configured, for example using suitable seals and valve design, to prevent unwanted air ingress and/or blood egress from the cartridge 1A. The microfluidic cartridge 1A may operate as a standalone cartridge or it may require an external device in order to operate, for example to actuate valves or other controllers, operate sensors, and record data.

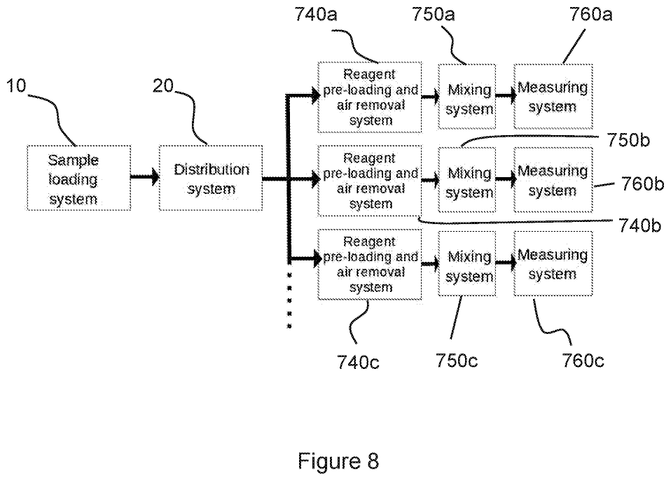

The distribution system 20 has the main function of receiving the blood sample injected into the loading system 10 and distributing the blood sample into a plurality of distribution channels 23 so that different types of measurements can be performed simultaneously by the device using different reagents or different types of measurement systems, as shown schematically in FIG. 8. In the embodiment of FIG. 8, the distribution system 20 receives sample fluid from the loading system 10 and distributes the sample fluid into at least three separate flow streams or portions. However, other embodiments of the distribution system 20 may have fewer than three or more than three separate flow streams or portions, the actual number of separate flow streams or portions being limited by space and fluid flow requirements. Each flow stream flows into a separate reagent pre-loading system 740a, 740b, 740c that in turn are arranged in fluid communication with separate mixers 750a, 750b, 750c and separate measurement systems 760a, 760b, 760c. Accordingly, by distributing the sample fluid into separate flow streams, different measurements of the sample fluid may be taken simultaneously in the measuring device 1.

In some embodiments, the distribution system 20 comprises of a distribution body that has six distribution channels 23 formed therein, shown schematically in FIG. 2. The distribution channels 23 may each lead towards a separate reagent pre-loading system 40 as shown schematically in FIG. 8. The distribution system 20 may include an overflow reservoir 24 in which any excess blood injected into the loading system 10 may pass into for collection in a separate container 26, shown schematically in FIG. 2. This functionality allows the blood to be loaded into the loading system 10 with a passive control over the volume of the sample rather than requiring an active control to load a precise volume, making the device more user friendly. The volume of sample fluid being loaded into the loading system 10 must be controlled so that the concentration of sample fluid to reagent is known and controlled and can therefore deliver a meaningful measurement result. The distribution system 20 further includes an air vent 28 associated with the overflow reservoir 24 for removing air contained in the fluid pathway upstream of the distribution channels 23 to be removed from the cartridge 1A. The air vent 28 and other air vents disclosed in the present disclosure may be in the form of a valve, including an air permeable valve. Accordingly, the features of the distribution system 20 may enhance the accuracy of the sample volume in a passive manner that facilitates ease of use as well as allowing multiple measurements to be performed simultaneously.

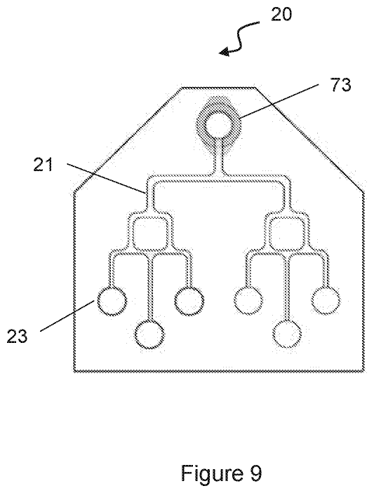

A cross-sectional view of an example of a splitter portion of the distribution body of the distribution system 20 is shown in FIG. 9. The air vent 28 and overflow reservoir 24 of the distribution system 20 are not visible in this view. The sample fluid enters the distribution system 20 at a distribution inlet 73. A cascade of bifurcating microfluidic channels 21 formed in the distribution body distributes the sample fluid into the six distribution channels 23. The volume of each of the channels may be below a pre-determined volume so as to minimise the amount of sample fluid utilised in the measurement. In one example, each channel may have a volume of below 50 μl. In use, all of the distribution channels 23 must be full of sample fluid with no air pockets nor dead volume generated. The distribution system of FIGS. 8 and 9 is especially beneficial for the simultaneous measurement of the sample fluid with different reagents, which is a feature that becomes particularly important when measuring time-sensitive fluids such as blood. However, the distribution system 20 is not essential for the functioning of the device. For example, if the tester only wishes to use a single reagent, the distribution device 20 may be omitted. It would also be possible to run multiple measurement devices without distribution systems 20, effectively running a manual distribution system.

If no distribution system is present, the sample fluid flows through loading system 10 directly through a sample fluid conduit 22 (effectively. a single distribution channel) towards the reagent pre-loading system 40. The term ‘sample fluid conduit’ 22 used herein can refer to the conduit that carries the sample fluid to the reagent pre-loading system 40, regardless of whether a distribution system 20 or other form of fluid processing or control is present in the measuring device 1.

As shown in FIG. 2, a check valve 29 is located in the sample fluid conduit 22 between the distribution system 20 and the reagent pre-loading system 40. The check valve 29 prevents backflow from the reagent pre-loading system 40 located downstream of the distribution system 20 so as to prevent sample loss and contamination between reagent-blood mixtures from different channels.

The reagent pre-loading system 40 has the purpose of introducing the reagent into the cartridge 1 for mixing with the sample fluid sample, e.g. blood. The embodiments of the reagent pre-loading system 40 of the present disclosure additionally substantially remove the air present in the reagent pre-loading system 40 as well as any air introduced into the cartridge 1A by the reagent or sample fluid loading into the cartridge 1A. This substantial removal of the air is an advantageous aspect of the reagent loading and mixing of the reagent with the sample fluid, as the presence of air between the sample fluid and reagent acts as a gap, preventing mixing between the sample and reagent. Throughout this disclosure, the term “reagent pre-loading system” refers to a system that is configured for receiving a reagent into the cartridge 1A prior to loading of a sample fluid into the reagent pre-loading system. This includes pre-loading at the time of manufacture of the cartridge 1A, but also allows the customisation of the cartridge 1A for a particular reagent at a later date. For example, the empty cartridge 1A may be customised later for a specific industry or purpose. In this case, the empty cartridge 1A may be provided by the cartridge manufacturer to a second manufacturer that requires a particular reagent for a particular testing purpose. The second manufacturer can add the reagent(s) that it requires into the cartridge for later use. Alternatively, a user selected reagent may be introduced into the cartridge 1A at the point of care or time of use, rather than being limited to the reagent being pre-determined and introduced into the cartridge 1A at the time of manufacture. Accordingly, the cartridge 1A can be customized for a desired use at any point prior to loading of the sample fluid into the cartridge 1A. The reagent pre-loading system 40 is configured for receiving a fluid reagent, more particularly a liquid reagent or a pre-hydrated dry reagent in liquid form, at a reagent admission arrangement of the reagent pre-loading system.

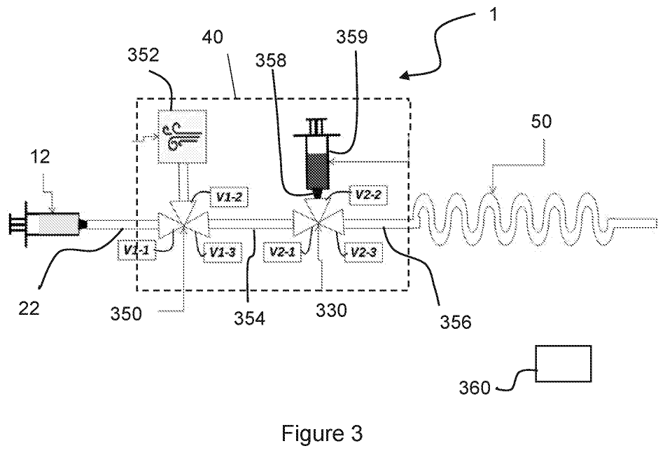

A partial schematic view of the measuring device 1 is shown in FIG. 3, focusing on the reagent pre-loading system 40. In order to control the admission and/or direction of the fluid injected into the cartridge 1A, a reagent admission arrangement and a sample admission arrangement are employed. The reagent admission arrangement may comprise a reagent valve arrangement 330. The sample admission arrangement may comprise a sample valve arrangement 350. In the embodiment of FIG. 3, the reagent valve arrangement 330 and the sample valve arrangement 350 each comprise of a three-way valve. The sample valve arrangement 350 has a first valve V1-1, a second valve V1-2 and a third valve V1-3. The reagent valve arrangement 330 has a first valve V2-1, a second valve V2-2 and a third valve V2-3.

The valve V1-1 of the sample valve arrangement 350 is operable to selectively open or close a sample fluid conduit 22 through which the fluid sample may pass from the distribution system 20 to the reagent pre-loading system 40. The valve V1-2 is operable to selectively open or close an air vent 352. The valve V1-3 is operable to selectively open or close a sample and reagent combination conduit 354 between the sample valve arrangement 350 and the reagent valve arrangement 330. The sample and reagent combination conduit 354 is a fluid pathway between the sample valve arrangement 350 and the reagent valve arrangement 330 in which the sample fluid and the reagent are combined together.

The valve V2-1 of the reagent valve arrangement 330 is operable to selectively open or close the sample and reagent combination conduit 354 between the sample valve arrangement 350 and the reagent valve arrangement 330 at the reagent loading system 40. The valve V2-2 is operable to selectively open or close fluid communication with a reagent delivery port 358 at which a liquid reagent is introduced into the cartridge 1A. The valve V2-3 is operable to selectively open or close fluid communication to the mixer conduit 356.

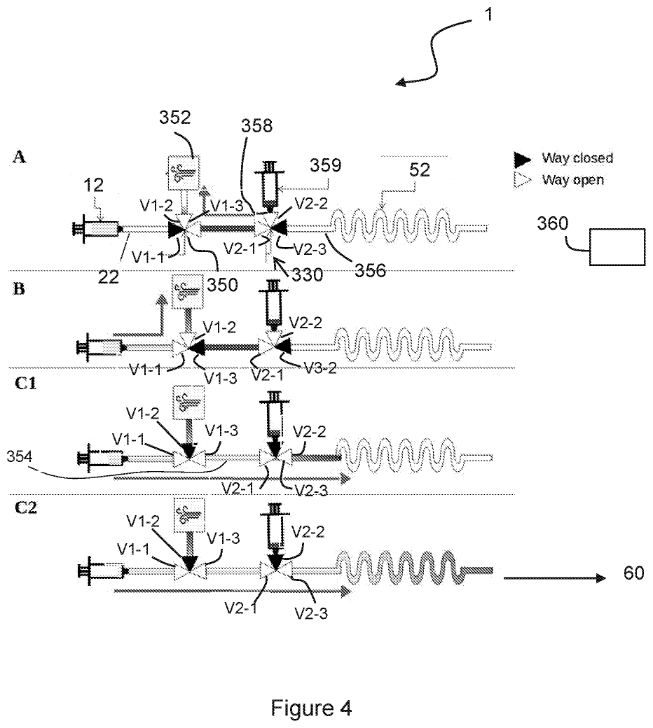

FIG. 4 is a schematic diagram of a series of process steps A, B, and C (shown in two stages C1 and C2) that are taken to operate the reagent valve arrangement 330 and the sample valve arrangement 350 in order to introduce the reagent and the blood sample into the cartridge 1A and to prevent ingress of air and remove air present in the cartridge 1A to thereby achieve good mixing of the blood sample and the reagent. Each of the steps A, B, and C require specific valve conditions that must be followed in sequence. In the initial state, the sample valve arrangement 350 is closed upstream towards the sample distribution conduit 22 at valve V1-1, whilst the reagent valve arrangement 330 is closed downstream at valve V2-3 to the mixer conduit 356 (see FIG. 4(A)). Accordingly, the sample and reagent combination conduit 354 is closed to fluid communication with sample fluid at the sample valve arrangement 350 and is closed at the reagent valve arrangement 330 to fluid communication with any fluid e.g. air present in the mixer conduit 356. The sample and reagent combination conduit is in fluid communication with the reagent delivery port 358 and the air vent 352. In this configuration, the reagent is pre-loaded into the cartridge 1A at the reagent delivery port 358, for example using a syringe 359 or other reagent delivery device as is known in the art. The reagent may be pre-loaded into the cartridge 1 at any point prior to loading the blood sample into the cartridge 1A. Valve V1-2 and valve V1-3 are open, as are valves V2-1 and V2-2. Accordingly, as the reagent is pre-loaded into the cartridge 1A, it flows into the sample and reagent combination conduit 354 and the air located in the sample and reagent combination conduit 354 between the two valve arrangements 350, 330 is substantially removed through the air vent 352 so as to be substantially eliminated from the sample and reagent combination conduit 354. The sample and reagent combination conduit 354 is thus primed with the reagent. The sample fluid will be admitted in to the sample and reagent combination conduit 354 into the leading front of reagent. As such, the sample fluid can be admitted into the sample and reagent combination conduit 354 and contact the reagent rather than air. The substantial removal of air present in the sample and reagent combination conduit 354 upon admission of the liquid reagent into the sample and reagent conduit 354 mitigates or substantially removes the possibility of an air pocket remaining in the sample and reagent combination conduit 354 ahead of the leading front of liquid reagent. At the end of this step, the sample and reagent conduit 354 may be completely filled with reagent.

After the reagent is loaded, in step B both the sample valve arrangement 350 and the reagent valve arrangement 330 are closed downstream at valves V1-3 and V2-3 respectively (see FIG. 4(B)) such that the sample and reagent conduit 354 is closed to the further ingress of air. Valve V1-1 at the upstream side of the sample valve arrangement 350 remains open. The reagent is in the sample and reagent combination conduit 354 with air having been substantially removed from the sample and reagent combination conduit 354 in the previous step. The cartridge I can remain in this state until the blood sample is ready to be loaded into the cartridge 1. This may occur at any time between shortly after manufacture of the cartridge 1 and immediately prior to sample loading. In this configuration, the blood is loaded into the cartridge 1A at the sample fluid port 14. The first portion of blood to enter the cartridge 1A flows through the sample fluid conduit 22 (via the distribution channels 23 if present) and expels (i.e. removes or substantially removes) the leading front of air through the sample valve arrangement 350 through the air vent 352 as well as any air present within the sample valve arrangement 350.

Once the sample valve arrangement 350 is primed with blood, in step C both the sample valve arrangement 350 and the reagent valve arrangement 330 are closed to the ambient atmosphere i.e. they are closed on their sides facing respectively the air vent 352 (V1-2) and the reagent delivery port 358 (V2-2) as shown in FIG. 4(C1). This step aims to prevent further air entering the sample valve arrangement 350 that may compromise the good mixing of the blood and the reagent. The valves V1-1, V1-3, V2-1 and V2-3 are all open, providing a fluid pathway directly from the sample distribution conduit 22 through the sample and reagent combination conduit 354 and into the mixer conduit 356 towards the mixer 52. In this valve configuration, the remaining blood is injected into the cartridge 1A at the sample fluid port 14, through the reagent pre-loading system 40 towards the mixer 52 at a suitable shear rate to assist good mixing, for example 7 s−1. The injected blood carries the reagent in the sample and reagent combination conduit 354 with it. The blood and reagent start mixing upstream of the mixer 52. Once they are in the mixer 52, the blood and reagent are mixed and delivered to the measuring system 60 as shown schematically in FIG. 4(C2).

It should be noted that steps B and C occur in quick succession. The valve conditions for the valves V1-1, V1-2, V1-3, V2-1, V2-2, V2-3 may be actively controlled by a controller 360 (shown schematically in FIG. 3) when an appropriate amount of blood has expelled the air in the sample valve arrangement 350 (as determined by time or by detection methods using one or more sensors, for example, but not limited to, strain gauge or vibration sensors). Alternatively, the valve conditions may be passively controlled by the use of passive activation, for example induced by pressure changes.

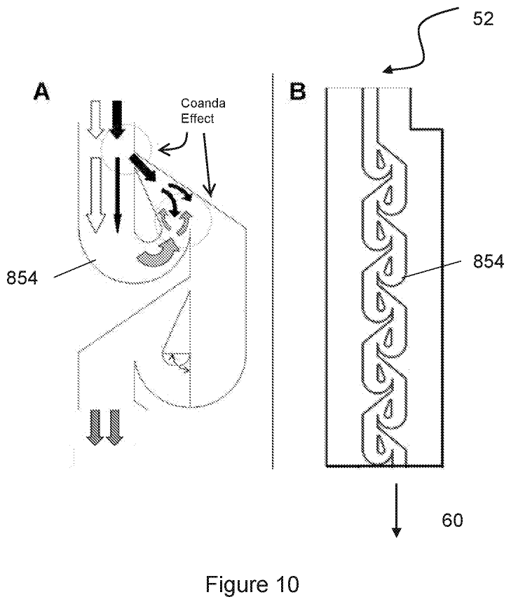

Referring again to FIG. 2, the mixing system 50 has the main function of fully mixing the blood flowing from the distribution channels 23 or sample fluid conduit 22 with the reagent that has been pre-loaded in the cartridge 1A via the reagent pre-loading system 40. The two fluids are fully mixed in the mixer 52 and carried into the measuring system 60. A check valve 54 at the end of the mixer 52 prevents backflow from the measuring system 60. The mixer 52 may be a tube of the measuring system 60 or it may be a separate mixer 52. The mixer 52 may, for example, be a micromixer comprising a zig-zag mixing channel as shown schematically in FIGS. 3 and 4. The micromixer may be of a type known as a ‘split and recombination’ micromixer in which the mixing channel utilises the Coanda effect to mix the two fluids. In an example embodiment, the mixer 52 may be able to mix the two fluids, e.g. blood and reagent, in less than 10 seconds and may have a dead volume of below 50 μl. However, as discussed elsewhere in this disclosure, these parameters are application specific and also depend on user preference. A longer mixing time may be acceptable for some applications whilst a shorter mixing time may be preferred in others. A schematic of a cross-section of a suitable micromixer 52 having a mixing channel 854 that utilises the Coanda effect is shown in FIG. 10B. An enlarged view of the mixing channel 854 of the mixer 52 and its operation in splitting and combining two fluids is shown in FIG. 10A. These types of mixing systems are well known in the art. The mixing channel shown in FIG. 9A is as published in C. C. Hong, J. W. Choi, and C. H. Ahn, “A novel in-plane passive microfluidic mixer with modified Tesla structures,” Lab Chip, vol. 4, no. 2, pp. 109-113, 2004, doi: 10.1039/b305892a. The measuring system 60 is located downstream of the mixing system 50. In some embodiments the cartridge 1A is formed integrally with the measuring system 60. Alternatively, the cartridge 1A may be formed separately to the measuring system 60. The measuring system 60 has a closed end, otherwise known as dead-ended, i.e. the mixed blood and reagent is contained within the measuring system and does not simply pass through it as in an open-ended system. A closed-end measuring system is configured to contain a controlled volume of the mixed sample fluid and reagent to be measured. The closed end or dead-ended system is used so as to minimise the amount of blood and reagent needed to conduct the measurements, and also to maintain control over the relative concentrations of the blood and the reagent that are measured. This requirement means that none of the blood used must be wasted, hence it is especially important that the blood sample and reagent are well mixed with no air pockets prior to measurement taking place. A closed ended (or dead-ended) system is especially important for some measuring systems, such as a rheometer, that are used to measure changes in the material properties with time. The leading front of blood entering the measuring system 60 expels any air within the measuring system 60 through air vents 64 as shown in FIG. 2 and as described below.

The measuring system 60 shown in FIG. 2 is configured for measuring the rheological properties of the sample fluid. The present applicant's earlier filed patent application PCT/GB2017/053393 discloses an example of such a measuring system 60 and is hereby incorporated by reference in its entirety. The measuring system 60 includes a manifold 62 and one or more pistons 63 positioned in a fluid conduit (not shown in FIG. 2), each piston including an auto valve 65 as shown in FIG. 2. An example of a suitable piston 63 and auto valve 65 arrangement is disclosed in the present applicant's earlier PCT application PCT/AU2020/050902, which is hereby incorporated by reference in its entirety. The auto valve 65 is a fluid check-valve having a hydrophilic porous material positioned upstream of a hydrophobic porous material relative to fluid flow. As blood or other sample fluid enters the measuring system 60, the leading front of blood pushes any air in the measuring system through the hydrophilic porous material and the hydrophobic porous material and out of the air vent 64. As blood proceeds to fill the measuring system 60, the hydrophilic material retains the liquid whilst hindering passage of air there through from downstream of the check-valve. The hydrophobic material hinders any blood that may pass through the hydrophilic porous material from passing through it. In the embodiment shown in FIG. 2, the measuring system 60 derives the rheology of the blood sample by measuring the pressure drop in the manifold 62 when a known flow is imposed. Whilst the present embodiment of the measuring system 60 is concerned with measuring rheological properties of blood, the measuring system 60 may be adapted for measuring other parameters such as platelet aggregometry, turbidity, or other optical, electrical or mechanical characteristics as is also known in the art.

A summary of the valve conditions for each of the valves V1-1, V1-2, V1-3 and V2-1, V2-2 and V2-3 during operation of the valves 350, 330 as described above and in relation to FIG. 4 is shown in Table 1 below.

TABLE 1

| | Sample valve arrangement | | | Reagent valve arrangement | | |
|---|---|---|---|---|---|---|
| | V1-1 | V1-2 | V1-3 | V2-1 | V2-2 | V2-3 |
| Step A | Closed (←) | Open (↑) | Open (←) | Open (←) | Open (↓) | Closed (→) |
| Step B | Open (→) | Open (↑) | Closed (→) | Open (←) | Open (↓) | Closed (→) |
| Step C | Open (→) | Closed (↑) | Open (→) | Open (→) | Closed (↑) | Open (→) |

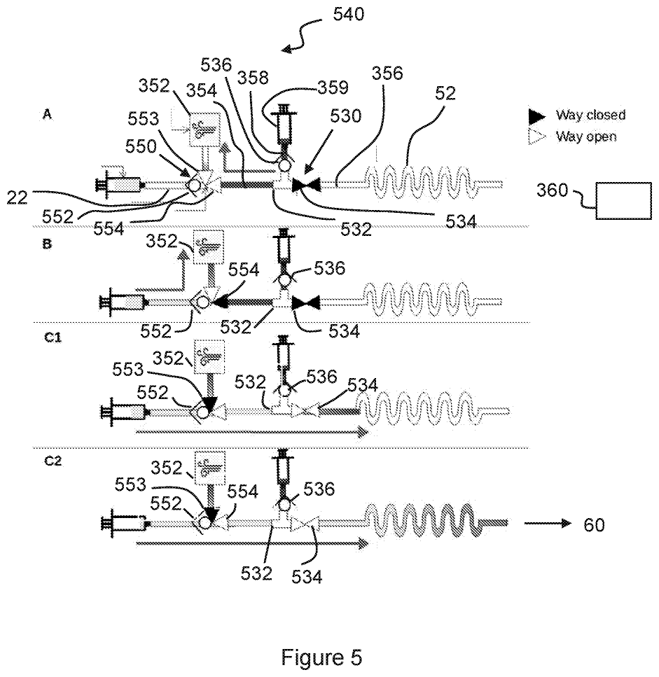

An alternative embodiment of the reagent pre-loading system 540 is shown in FIG. 5. This embodiment is structurally similar to the reagent loading system 40, with an alternative sample admission arrangement in the form of a sample valve arrangement 550 and a reagent admission arrangement in the form of a reagent valve arrangement 530. The sample valve arrangement 550 and the reagent valve arrangement 530 are designed based on the valve conditions during operation of the reagent loading system 540.

For example, V2-1 is always open. Accordingly, the reagent valve arrangement 530 need not be a three way valve but may be replaced by a T-junction conduit 532 connected upstream to the sample and reagent combination conduit 354 and the sample valve arrangement 550 and connected downstream to a 2-way valve 534 that selectively opens and closes fluid access to the mixer 52 via the mixer conduit 356. At the same time, valve V2-2 may be replaced by a check valve 536 at the reagent delivery port 358 so as to permit substantially one way fluid flow from the reagent delivery port 358 into the T-junction conduit 532.

Similarly, for the sample valve arrangement 550, valve V1-1 may be replaced by a check valve 552 that permits substantially one way fluid flow only from the sample distribution channel or distribution channel 22 to the T-junction conduit 532. Valve V1-2, V1-3 of the sample valve arrangement 550 is a two way valve 554 that is operable to selectively open or close the air vent 352 and the sample and reagent combination conduit 354 to the reagent valve arrangement 530, to allow air in the sample and reagent combination conduit to be expelled (i.e. substantially removed or removed) through the air vent 352, for example to the atmosphere. Alternatively, valve V1-2 may be a gas-selective check valve that will permit air to pass from either the reagent side (from the sample and reagent combination conduit 354) or from the sample side (from the sample distribution channel 22), but will not allow air to return into the sample valve arrangement 550 or back into the sample and reagent combination conduit 354.

The operation of the reagent loading system 540 is shown in Table 2 below and in FIG. 5 as a series of process steps A, B, and C (shown in two stages C1 and C2) that are taken to operate the sample valve arrangement 550 and the reagent valve arrangement 530 in order to introduce the reagent and the blood sample into the cartridge 1A and to prevent ingress of air and remove air present in the sample and reagent combination conduit 354 of the cartridge 1A to thereby achieve good mixing of the blood sample and the reagent. Each of the steps A, B, and C require specific valve conditions that must be followed in sequence. The sequence is functionally the same as for the reagent loading system 40.

TABLE 2

| | Sample valve arrangement | | | Reagent valve arrangement | | |
|---|---|---|---|---|---|---|
| | V1-1(552) | V1-2(553) | V1-3 (554) | No valve | V2-2 (536) | V2-3 (534) |
| Step A | Closed (←) | Open (↑) | Open (←) | Open (←) | Open (↓) | Closed (→) |
| Step B | Open (→) | Open (↑) | Closed (→) | Open (←) | Open (↓) | Closed (→) |
| Step C1 | Open (→) | Closed (↑) | Open (→) | Open (→) | Closed (↑) | Open (→) |
| Step C2 | Open (→) | Closed (↑) | Open (→) | Open (→) | Closed (↑) | Open (→) |

In the initial state of step A, the sample valve arrangement 550 is closed upstream towards the sample distribution conduit 22 at the check valve 552, whilst the reagent valve arrangement 530 is closed downstream at valve 534 to the mixer conduit 356 (see FIG. 5(A)). In this configuration, the reagent is pre-loaded into the cartridge 1A at the reagent delivery port 358, for example using a syringe 359 or other reagent delivery device. The reagent may be pre-loaded into the cartridge 1A at any point prior to loading the blood sample into the cartridge 1A. The T-junction conduit 532 is open to the sample and reagent combination conduit 534 and the two way valve 554 is open at valve 553 to permit fluid flow from the sample and reagent combination conduit 354 through the air vent 352. The check valve 536 is open to permit reagent to enter the reagent delivery port 358. Accordingly, as the reagent is pre-loaded into the cartridge 1A, it flows into the sample and reagent combination conduit 354 and the air located in the sample and reagent combination conduit 354 between the two valve arrangements 550, 530 is displaced by the reagent and substantially removed through the air vent 352 so as to be substantially eliminated from the sample and reagent combination conduit 354. The sample and reagent combination conduit 354 is thus primed with the reagent. The sample fluid will be admitted in to the sample and reagent combination conduit 354 into the leading front of reagent. As such, the sample fluid can be admitted into the sample and reagent combination conduit 354 and contact the reagent rather than air.

After the reagent is loaded, in step B both the sample valve arrangement 550 and the reagent valve arrangement 530 are closed downstream at two valves 554 and 534 respectively (see FIG. 5(B)) such that the sample and reagent conduit 554 is closed to the further ingress of air. The reagent is in the sample and reagent combination conduit 354 with air having been substantially removed from the sample and reagent combination conduit in the previous step. The cartridge 1A can remain in this state until the blood sample is ready to be loaded into the cartridge 1A. In this configuration, the blood is loaded into the cartridge 1A at the sample fluid port 14. The first portion of blood to enter the cartridge 1A flows through the sample fluid conduit 22 (via the distribution channels 23 if present) and expels the leading front of air through the sample valve arrangement 550 through the air vent 352 as well as any air present within the sample valve arrangement 550.

Once the sample valve arrangement 550 is primed with blood, in step C both the sample valve arrangement 550 and the reagent valve arrangement 530 are closed to the ambient atmosphere i.e. they are closed on their sides facing respectively the air vent 352 (at valve 553 of the sample valve arrangement 550) and the reagent delivery port 358 (at valve 534 of the reagent valve arrangement 530) as shown in FIG. 5(C1). The check valve 552 and the downstream side of valve 554 are open, as is the two way valve 534, providing a fluid pathway directly from the sample distribution conduit 22 through the sample and reagent combination conduit 354 and into the mixer conduit 356 towards the mixer 52. In this valve configuration, the remaining blood is injected into the cartridge 1A at the sample fluid port 14 towards the mixer 52, at a suitable shear rate to assist good mixing, for example 7 s⁻¹. The injected blood carries the reagent in the sample and reagent combination conduit 354 with it. The blood and reagent may start mixing upstream of the mixer conduit 356. Some mixing may take place in the mixer conduit 356, however the majority of the mixing may take place in the mixer 52. The blood and reagent are mixed and delivered to the measuring system 60 as shown schematically in FIG. 5(C2).

The valve conditions for the valves 552, 553, 554, 534 and 536 may be actively set and changed by the controller 360 (shown schematically in FIG. 3) when an appropriate amount of blood has expelled the air in the sample valve arrangement 550 (as determined by time or by detection methods as will be apparent to the skilled person) or they may be controlled passively by the use of passive activation, for example induced by pressure changes.

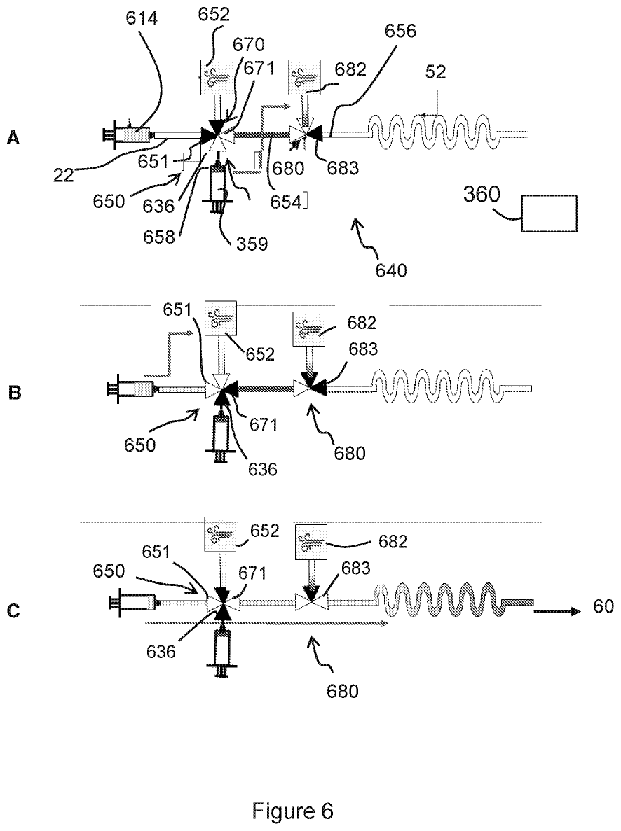

FIG. 6 shows a further embodiment of the reagent pre-loading system 640. This embodiment is structurally similar to the reagent loading system 40, with an alternative sample admission arrangement in the form of sample valve arrangement 650 and a reagent admission arrangement in the form of reagent valve arrangement 630. The sample valve arrangement 650 and the reagent valve arrangement 630 are designed based on the valve conditions during operation of the reagent loading system 640.

The reagent valve arrangement 630 and the sample valve arrangement 650 form at least part of a four-way valve arrangement 670 configured to selectively admit reagent and sample fluid or other material into the sample and reagent combination conduit 654. The reagent valve arrangement 630 includes a valve or check valve 636 for selective admission of reagent at the reagent delivery port 658 so as to permit substantially one way fluid flow from the reagent delivery port 658 into the sample and reagent combination conduit 654. The sample valve arrangement 650 similarly includes a valve or check valve 651 for selective admission of sample fluid at the sample fluid conduit 22 to permit substantially one way fluid flow from the sample fluid conduit 22 into the sample and reagent combination conduit 654. The four-way valve arrangement 670 also includes an air vent or valve 652 and a downstream side valve 671 that controls access of sample fluid and reagent into the sample and reagent combination conduit 654.

The system 640 further includes a downstream valve arrangement 680 configured for selective expulsion of air in the sample and reagent combination conduit 654 upon admission of reagent therein. It includes an air vent or valve 682. The downstream valve arrangement 680 further includes a downstream side valve 683 that is configured to selectively permit combined sample fluid and reagent in the sample and reagent combination conduit 654 to pass into the mixer 52, optionally via a mixer conduit 656. The downstream valve arrangement 680 is a three-way valve providing selective fluid communication between the sample and reagent combination conduit 654, the air vent or valve 682 and the mixer 52.

The operation of the reagent loading system 640 is shown in FIG. 6 as a series of process steps A, B, and C that are taken to operate the sample valve arrangement 650 and the downstream valve arrangement 680 in order to introduce the reagent and the blood sample into the cartridge 1A and to prevent ingress of air and remove air present in the cartridge 1A to thereby achieve good mixing of the blood sample and the reagent. Each of the steps A, B, and C require specific valve conditions that must be followed in sequence. The sequence is functionally the same as for the reagent loading systems 40, 540.

In the initial state of step A, the sample valve arrangement 650 is closed upstream towards the sample distribution conduit 22 at the valve or check valve 651, whilst the reagent valve 636 is open to admit reagent into the sample and reagent combination conduit 654. The downstream side valve 671 is open for this purpose. The air vent or valve 652 is closed. The downstream valve arrangement 680 is closed to the mixer conduit 656 at the downstream side valve 683 (see FIG. 6(A)), and open to both the sample and reagent combination conduit 654 and the air vent or valve 682. In this configuration, the reagent is pre-loaded into the cartridge 1A at the reagent delivery port 658, for example using a syringe 359 or other reagent delivery device. The reagent may be pre-loaded into the cartridge 1A at any point prior to loading the blood sample into the cartridge, as with previous embodiments. The downstream side valve 671 of the sample admission valve 650 is open to the sample and reagent combination conduit 654 whilst the downstream side valve 683 of the downstream valve 680 is closed to the mixer conduit 656. The valve or check valve 636 is open to permit reagent to enter the reagent delivery port 658. Accordingly, as the reagent is pre-loaded into the cartridge 1A, it flows into the sample and reagent combination conduit 654 and the air located in the sample and reagent combination conduit 654 between the four way valve 670 and the downstream valve arrangement 680 is expelled i.e. removed or substantially removed through the air vent 682 so as to be substantially eliminated from the sample and reagent combination conduit 654. The sample and reagent combination conduit 654 is thus primed with the reagent. The sample fluid will be admitted in to the sample and reagent combination conduit 654 into the leading front of reagent. As such, the sample fluid can be admitted into the sample and reagent combination conduit 654 and contact the reagent rather than air.

After the reagent is loaded, in step B the check valve 636 and the downstream side valve 671 of the four way valve 670 are closed. The check valve 651 of the sample valve arrangement 650 is opened, together with the air vent or valve 652 (see FIG. 6(B)). The downstream valve arrangement 680 remains open to the reagent and sample fluid combination conduit 654, but the air vent or valve 682 is now closed. The reagent is in the sample and reagent combination conduit 654 and the sample and reagent conduit 354 is closed to the further ingress of air. The cartridge 1A can remain in this state until the blood sample is ready to be loaded into the cartridge 1A. In this configuration, the blood is loaded into the cartridge 1 at the sample fluid port 614 to prime the sample valve arrangement 650 with blood. The first portion of blood to enter the cartridge 1A expels, i.e. substantially removes, the leading front of air through the sample valve arrangement 650 of the four way valve 670 through the air vent 652, as well as any air present within the sample valve arrangement 650.

Once the sample valve arrangement 650 of the four way valve 670 is primed with blood, in step C both the air vent 652 and the air vent 682 are closed as shown in FIG. 6(C). The downstream side valve 671 is open to the sample and reagent combination conduit 654 and the downstream valve arrangement 680 is also open to both the sample and reagent combination conduit 654 and the mixer conduit 656 at valve 683, providing a fluid pathway directly from the sample distribution conduit 22 through the sample and reagent combination conduit 654 and into the mixer conduit 656 towards the mixer 52. In this valve configuration, the remaining blood is injected into the cartridge 1A at the sample fluid port 614 towards the mixer 52, at a suitable shear rate to assist good mixing, for example 7 s−1. The injected blood carries the reagent in the sample and reagent combination conduit 654 with it. The blood and reagent start mixing upstream of the mixer 52. Once they are in the mixer 52, the blood and reagent are mixed and delivered to the measuring system 60 as shown schematically in FIG. 6(C).

TABLE 3

| | | | | | Downstream valve | |
|---|---|---|---|---|---|---|
| | Four-way valve arrangement | | | | arrangement | |
| | 651 | 652 | 636 | 671 | 682 | 683 |
| Step A | Closed (←) | Closed (↑) | Open (←) | Open (←) | Open (↓) | Closed (→) |
| Step B | Open (→) | Open (↑) | Closed (→) | Closed (←) | Closed (↓) | Closed (→) |
| Step C | Open (→) | Closed (↑) | Open (→) | Open (→) | Closed (↑) | Open (→) |

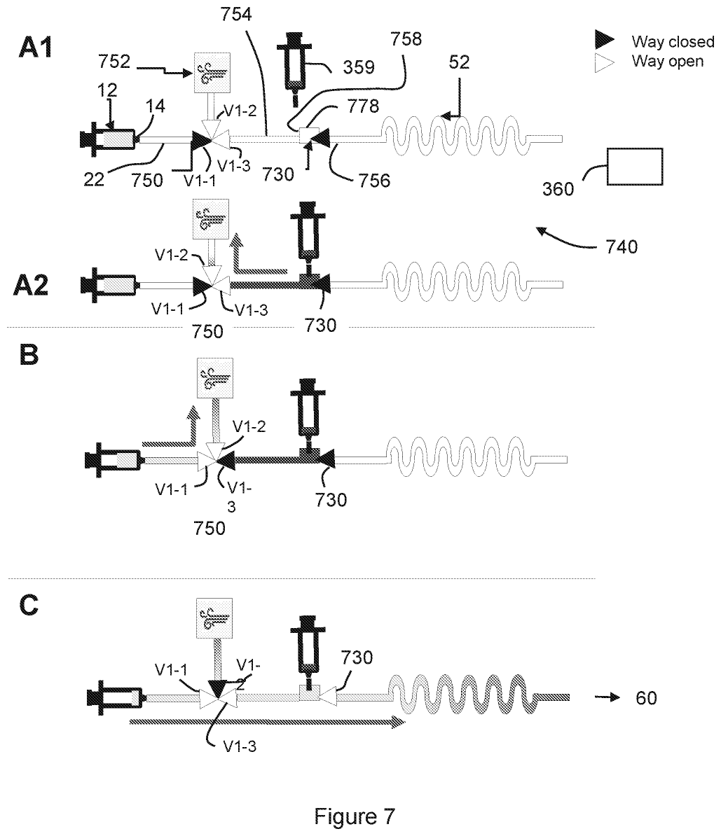

In a further embodiment of the reagent pre-loading system 740, shown schematically in FIG. 7, the reagent delivery port 758 comprises a sealed membrane 778. The sealed membrane 778 covers and seals the reagent port 758 and may be pierced with a syringe 359 containing the reagent, to admit reagent into the reagent delivery port 758 at which the reagent is delivered into the cartridge 1A. In this embodiment, the reagent admission arrangement in the form of the reagent valve arrangement 730 includes a single valve for controlling fluid flow from the reagent delivery port 758 and/or the sample and reagent combination conduit 754 into the mixer 52. The sample admission arrangement in the form of the sample valve arrangement 750 is identical to the sample valve arrangement 350 and comprises of a three-way valve. The sample valve arrangement 750 has a first valve V1-1, a second valve V1-2 and a third valve V1-3.

The valve V1-1 of the sample valve arrangement 750 is operable to selectively open or close a sample fluid conduit 22 through which the fluid sample may pass from the distribution system 20 or directly from the sample fluid port 14. The valve V1-2 is operable to selectively open or close an air vent 752. The valve V1-3 is operable to selectively open or close the sample and reagent combination conduit 754 between the sample valve arrangement 750 and the reagent valve arrangement 730.

The sealed membrane 778 may be pierced to selectively open fluid communication with the reagent delivery port 758 at which reagent is introduced into the reagent pre-loading system 40. The single valve of the reagent valve arrangement 730 is operable to selectively open or close fluid communication to the mixer conduit 756 and/or the mixer 52.

FIG. 7 is a schematic diagram of a series of process steps A1, A2, B, and C that are taken to operate the reagent valve arrangement 730 and the sample valve arrangement 750 in order to introduce the reagent and the blood sample into the reagent pre-loading system 740 and to prevent ingress of air and remove air present in the cartridge to thereby achieve good mixing of the blood sample and the reagent. Each of the steps A1, A2, B, and C require specific valve conditions that must be followed in sequence. In the initial state (see FIG. 7(A1)), the sample valve arrangement 750 is closed upstream towards the sample distribution conduit 22 at valve V1-1, whilst the reagent valve arrangement 730 is closed to the mixer conduit 756. The reagent delivery port 758 is sealed by the sealed membrane 778 as shown in FIG. 7(A1). In this configuration, the reagent is pre-loaded into the cartridge 1A at the reagent delivery port 758 by piercing the sealed membrane 778 using the syringe 359 or other reagent delivery device as is known in the art. As shown in FIG. 7(A2), the reagent may then be injected through the pierced membrane 778 into the sample and reagent combination conduit 754. The reagent may be pre-loaded into the cartridge 1 at any point prior to loading the blood sample into the cartridge. Valve V1-2 and valve V1-3 are open; the reagent valve arrangement 730 is closed to fluid flow. Accordingly, as the reagent is pre-loaded into the cartridge 1A, it flows into the sample and reagent combination conduit 754 and air located in the sample and reagent combination conduit 754 between the two valve arrangements 750, 730 is substantially removed through the air vent 752 so as to be substantially eliminated from the sample and reagent combination conduit 754. The sample and reagent combination conduit 754 is thus primed with the reagent. The sample fluid will be admitted in to the sample and reagent combination conduit 754 into the leading front of reagent. As such, the sample fluid can be admitted into the sample and reagent combination conduit 754 and contact the reagent rather than air. At the end of this step, the sample and reagent conduit 754 is substantially completely filled with reagent. The cartridge 1 can remain in this state until the blood sample is ready to be loaded into the cartridge 1, however in this embodiment the reagent may typically be admitted into the reagent delivery port 758 immediately prior to sample loading.

After the reagent is loaded, in step B the sample valve arrangement 750 is closed downstream at valve V1-3 and the reagent valve arrangement 730 remains closed to the mixer 52 (see FIG. 7(B)). Valve V1-1 at the upstream side of the sample valve arrangement 750 can now be opened. The reagent is in the sample and reagent combination conduit 754 and the sample and reagent conduit 754 is closed to the further ingress of air. In this configuration, the blood is loaded into the cartridge 1A at the sample fluid port 14. The first portion of blood to enter the cartridge 1A flows through the sample fluid conduit 22 (via the distribution channels 23 if present) and expels the leading front of air through the sample valve arrangement 750 through the air vent 752 as well as any air present within the sample valve arrangement 750.

Once the sample valve arrangement 750 is primed with blood, in step C the valve V1-2 of the sample valve arrangement 750 is closed to the air vent 752 as shown in FIG. 7(C). The reagent valve arrangement 730 is now opened. The valves V1-1, V1-3 are also opened, providing a fluid pathway directly from the sample fluid conduit 22 through the sample and reagent combination conduit 754 and into the mixer conduit 756 towards the mixer 52. In this valve configuration, the remaining blood is injected into the cartridge 1A at the sample fluid port 14, through the reagent pre-loading system 40 towards the mixer 756 at a suitable shear rate to assist good mixing, for example 7 s−1. The injected blood carries the reagent in the sample and reagent combination conduit 754 with it. The blood and reagent may start mixing upstream of the mixer 52. Once they are in the mixer 52, the blood and reagent may be mixed and delivered to the measuring system 60 as shown schematically in FIG. 7(C).

It should be noted that steps B and C occur in quick succession, with the valve conditions either being actively changed by a controller 760 (shown schematically in FIG. 7A) when an appropriate amount of blood has expelled the air in the sample valve arrangement 750 (as determined by time or by detection methods using one or more sensors, for example, but not limited to, strain gauge or vibration sensors) or by the use of passive activation, for example induced by pressure changes.

The valve conditions for the valves 350, 330, 552, 553, 554, 534, 536, 630, 650, 680, 683, 730, 750 may be either actively set and changed by the controller 360 (shown schematically in FIGS. 3 to 7) when an appropriate amount of blood has expelled the air in the sample valve arrangement 550, 650, 750 (as determined by time or by detection methods as will be apparent to the skilled person) or by the use of passive activation, for example by induced by pressure changes.

The valves of the sample valve arrangements 350, 550, 650, 750 and the reagent valve arrangements 330, 530, 630, 730 may be replaced by electronic valves and/or pressure induced valves or mechanically actuated (e.g. magnetically or electrically), electrochemically actuated or hydraulically actuated valve as further alternative embodiments of those arrangements. The alternative configurations of the valve arrangements described here may increase the speed of the process and/or make it more user friendly. The footprint of the measuring device I may also be reduced. However, it will be apparent to the skilled person that other similar configurations could also be applied while maintaining the same process functionality, whereby fluid paths can be controlled at the appropriate stages within the process of operating the reagent loading system 40, 540, 640.

The embodiments of the reagent pre-loading system and measuring device disclosed herein are suitable for use with sample materials other than blood, for example other newtonian and non-newtonian fluids or liquid slurries such as mineral tailings and wastewater. Slurries such as these as the sample fluid may be mixed with different flocculants as reagents to determine the effect on the rheological properties of the liquid slurry. In this manner, the measuring device I can be used to determine the best flocculant type, structure etc to use to improve the operation and efficiency of processes such as thickeners, settlement ponds, or other gravity-based separation processes. As with blood and its reagents, the concentration of mineral tailings and flocculant needs to be controlled in order for the measurement to be meaningful. The dead-ended measuring device 1 of the present disclosure is therefore particularly suited for this use. Furthermore, the measuring device I can be used on site as a sample of the slurry is obtained, saving the cost, time and energy requirements of having to conduct measurements away from the site.

In some embodiments, the reagent pre-loading system 40 can be configured to also provide mixing between the sample and the reagent. Such mixing could occur through the incorporation of geometric features to promote mixing within the sample and reagent combination conduit 354, 654, 754. For example, geometric features may be added to promote the Coanda effect, or a Herringbone mixer, or they may form baffles. Alternatively, or in addition, mixing between the sample and the reagent may take place through a recirculation process with priming to expel air. For example, a recirculation conduit 800 may be added between the sample valve arrangement 350, 650, 750 and the reagent admission arrangement 330, 630, 730 of any one of the disclosed embodiments of the reagent pre-loading system 40, 540, 640. The recirculation conduit 800 may form a mixing loop with the sample and reagent combination conduit 354. In this embodiment the sample fluid is admitted into the recirculation conduit 800. Once the reagent and the sample fluid are admitted as described in the earlier embodiments, the sample valve arrangement 350, 650, 750 may be configured to permit fluid communication between the recirculation conduit 800 and the sample and reagent combination conduit 354. Mixing of the sample and the reagent may then take place prior to the mixed sample and reagent passing to the measuring system 60. A valve 880 is present in the recirculation conduit for selectively permitting the mixed sample fluid and reagent to pass to the measuring system 60.

In this disclosure, the term ‘reagent’ may include a chemical, reaction, reactant, chemoeffector, indicator, or testing agent. The term ‘mixed’ includes substantially mixed. The term ‘conduit’ may include a pipe, tube or other means of transporting fluid from one place to another. The term ‘air vent or valve’ may include an air permeable valve such as an air permeable membrane. The term ‘valve’ is understood as a means for controlling or stopping flow of material through an entry, exit, pipe, tubing, or conduit.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A reagent pre-loading system for a measuring device, comprising:
a reagent admission arrangement arranged for receiving a reagent into the reagent pre-loading system;
a sample admission arrangement arranged for receiving a sample fluid to be measured into the reagent pre-loading system,
wherein the reagent admission arrangement and the sample admission arrangement are each arranged in selective fluid communication with a sample and reagent combination conduit for combining the sample fluid and the reagent;
wherein the sample admission arrangement and the reagent admission arrangement are arranged to selectively: whilst the sample and reagent combination conduit is closed to fluid communication with the sample fluid at the sample admission arrangement, receive the reagent into the sample and reagent combination conduit so as to expel air from the sample and reagent combination conduit as the reagent is received therein and prime the sample and reagent combination conduit with the reagent; whilst the sample and reagent combination conduit is closed to fluid communication with the sample admission arrangement, receive the sample fluid into the sample admission arrangement so as to expel air from the sample admission arrangement and prime the sample admission arrangement with the sample fluid; and, whilst the sample and reagent combination conduit is open to fluid communication with the sample admission arrangement, admit further sample fluid into the sample admission arrangement and into the sample and reagent combination conduit for combining with the reagent.

2. The reagent pre-loading system of claim 1, wherein at least one of the reagent admission arrangement and the sample valve arrangement includes an air vent or valve.

3. The reagent pre-loading system of claim 1 or claim 2,
wherein the reagent admission arrangement comprises a reagent valve arrangement configured for selective admission of the reagent into the reagent pre-loading system; and
wherein the sample admission arrangement comprises a sample valve arrangement configured for selective admission of the sample fluid to be measured into the reagent pre-loading system.

4. The reagent pre-loading system of claim 2 or claim 3, wherein
the reagent valve arrangement is configured for selective fluid communication between at least a reagent delivery port and the sample and reagent combination conduit.

5. The reagent pre-loading system of claim 4, wherein the reagent valve arrangement is further configured for selective fluid communication between the sample and reagent combination conduit and a mixer.

6. The reagent pre-loading system of any one of claim 3 to claim 5, further comprising a sample fluid conduit arranged for receiving the sample fluid to be measured into the sample valve arrangement; wherein
the sample valve arrangement is configured for selective fluid communication between each of the sample fluid conduit, the sample and reagent combination conduit and the air vent or valve.

7. The reagent pre-loading system of any one of claims 3 to 6, configured to, upon admitting the reagent into the sample and reagent combination conduit, substantially remove air from the sample and reagent combination conduit.

8. The reagent pre-loading system of claim 7, wherein admitting the reagent into the sample and reagent combination conduit and removing air from the sample and valve combination conduit comprises: selectively admitting the reagent at the reagent delivery port into the sample and reagent combination conduit whilst the sample valve arrangement is closed to the sample fluid conduit and open to the air vent or valve, such that admission of the reagent into the sample and reagent combination conduit displaces air in the sample and reagent combination conduit and expels it through the air vent or valve.

9. The reagent pre-loading system of any one of claims 3 to 8, wherein the admitting the sample fluid so as to expel air from the sample valve arrangement and prime the sample valve arrangement with the sample fluid comprises: closing the sample valve arrangement to the sample and reagent combination conduit and opening the sample valve arrangement to the sample fluid conduit so as to selectively admit sample fluid into the sample fluid conduit, such that admission of the sample fluid into the sample fluid conduit displaces air in the sample valve arrangement and expels it through the air vent or valve.

10. The reagent pre-loading system of claim 9 when dependent on claim 5, wherein admitting further sample fluid into the sample valve arrangement and into the sample and reagent combination conduit for combining with the reagent comprises, whilst the reagent valve arrangement is open to the mixer and closed to the reagent delivery port: closing the sample valve arrangement to the air vent or valve and opening the sample valve arrangement to the sample and reagent combination conduit, such that further sample fluid delivered into the sample fluid conduit is admitted into the sample and reagent combination conduit for combining with the reagent in the sample and reagent combination conduit.

11. The reagent pre-loading system of any one of claims 6 to 10, wherein the sample valve arrangement comprises a three-way valve that is controllable to selectively open and/or close fluid access to and/or from the sample fluid conduit, the sample and reagent combination conduit and an air vent or valve.

12. The reagent pre-loading system of any one of claims 4 to 11 wherein the reagent valve arrangement comprises a three-way valve that is controllable to selectively open and/or close fluid access to and/or from the reagent delivery port, the sample valve arrangement via the sample and reagent combination conduit, and the mixer.

13. The reagent pre-loading system of any one of claims 4 to 11, wherein the reagent valve arrangement comprises a T-junction conduit arranged in fluid communication with the sample and reagent combination conduit at an upstream end thereof and in fluid communication with a mixer valve at a downstream end thereof, the mixer valve providing selective fluid communication between the T-junction conduit and the mixer; and a check valve adapted to permit substantially one way fluid flow from the reagent delivery port into the T-junction conduit.

14. The reagent pre-loading system of any one of claims 6 to 10 or claim 13, wherein the sample valve arrangement comprises a two-way valve providing selective fluid communication between the sample and reagent combination conduit and the air vent or valve; and a one way valve adapted to permit substantially one way fluid flow from the sample fluid conduit into the sample and reagent combination conduit and/or the air vent or valve.

15. The reagent pre-loading system of claim 13, wherein the mixer valve is selected from one of a pressure-induced valve, mechanically active valve, externally controlled pneumatic valve, burst valve or electronic valve.

16. The reagent pre-loading system of claim 14 or claim 15, wherein the two-way valve of the sample valve arrangement comprises a pressure-induced or electronic valve.

17. The reagent pre-loading system of any one of claims 14 to claim 16 wherein the one way valve comprises a check valve.

18. The reagent pre-loading system of claim 11 or claim 12 wherein the three-way valve comprises a pressure-induced or electronic valve.

19. The reagent pre-loading system of any one of claims 3 to 18, wherein the reagent valve arrangement and the sample valve arrangement are arranged in selected fluid communication via the sample and reagent combination conduit.

20. The reagent pre-loading system of claim 3, wherein the reagent valve arrangement and the sample valve arrangement form at least part of a four-way valve arrangement configured to selectively admit reagent and sample fluid into the sample and reagent combination conduit.

21. The reagent pre-loading system of claim 20, further comprising a downstream valve arrangement configured for selective expulsion of air in the sample and reagent combination conduit upon admission of reagent therein.

22. The reagent pre-loading system of claim 21, wherein the downstream valve arrangement is selectively configured to permit combined sample fluid and reagent in the sample and reagent combination conduit to pass into a mixer.

23. The reagent pre-loading system of claim 22, wherein the downstream valve arrangement is a three-way valve providing selective fluid communication between the sample and reagent combination conduit, an air vent or valve, and the mixer.

24. The reagent pre-loading system of any one of claims 20 to 22, wherein the four-way valve is an electronic valve or a pressure induced, mechanically actuated or hydraulically actuated valve.

25. The reagent pre-loading system of any one of claims 21-24, wherein the downstream valve is an electronic valve or a pressure induced valve.

26. The reagent pre-loading system of any one of claims 3 to 25, further comprising a controller configured to selectively operate one or more components of the sample valve arrangement and the reagent valve arrangement.

27. The reagent pre-loading system of claim 1, comprising a reagent delivery port, wherein the reagent admission arrangement comprises a pierceable sealed membrane arranged to sealingly close the reagent delivery port to fluid flow there through until it is pierced, and, upon the sealed membrane being pierced, to receive the reagent in the reagent pre-loading system.

28. The reagent pre-loading system of claim 1 or claim 27, wherein the reagent admission arrangement comprises a valve configured for selective fluid communication between the sample and reagent combination conduit and a mixer.

29. The reagent pre-loading system of any one of claim 1 or 27 or 28, wherein the sample admission arrangement is configured for selective fluid communication between each of a sample fluid conduit, the sample and reagent combination conduit and an air vent or valve.

30. The reagent pre-loading system of any one of claims 1 or 27 to 29 configured to, upon receiving the reagent into the sample and reagent combination conduit, substantially remove air from the sample and reagent combination conduit.

31. The reagent pre-loading system of claim 29 or claim 30, wherein the sample admission arrangement comprises a valve arrangement for selective admission of sample fluid into the reagent pre-loading system and selective fluid communication between the sample fluid conduit and the sample and reagent combination conduit, wherein the receiving the sample fluid so as to expel air from the sample admission arrangement and prime the sample admission arrangement with the sample fluid comprises: closing the sample admission arrangement to the sample and reagent combination conduit and opening the sample admission arrangement to the sample fluid conduit so as to selectively admit sample fluid into the sample fluid conduit, such that admission of the sample fluid into the sample fluid conduit displaces air in the sample admission arrangement through the air vent or valve.

32. The reagent pre-loading system of claim 31 when dependent upon claim 28 and claim 29, wherein admitting further sample fluid into the sample admission arrangement and into the sample and reagent combination conduit for combining with the reagent comprises, whilst the reagent admission arrangement is open to the mixer and closed to the reagent delivery port: closing the sample admission arrangement to the air vent or valve and opening the sample admission arrangement to the sample and reagent combination conduit, such that further sample fluid delivered into the sample fluid conduit is admitted into the sample and reagent combination conduit for combining with the reagent in the sample and reagent combination conduit.

33. A cartridge for a measuring device, comprising the reagent pre-loading system of any one of the preceding claims.

34. A measuring device for measuring a material property, comprising a sample loading port, a reagent pre-loading system arranged in selective fluid communication with the sample loading port, and a closed-ended measuring system arranged in fluid communication with the reagent pre-loading system, whereby the closed-ended measuring system is configured to contain a controlled volume of the sample fluid and reagent to be measured.

35. The measuring device of claim 34, further comprising a mixer arranged in fluid communication between the reagent pre-loading system and the measuring system.

36. The measuring device of claim 34 or claim 35, further comprising a distribution device arranged in selective fluid communication with the sample loading port and adapted for distributing a sample fluid introduced into the sample loading port to a plurality of distribution channels; and a plurality of the reagent pre-loading systems, each of the plurality of the reagent pre-loading systems being arranged in fluid communication with a respective one of the plurality of distribution channels for receiving a portion of the sample fluid for combining with a reagent.

37. The measuring device of claim 36, wherein the distribution device comprises a distributing body having a sample fluid inlet arranged in fluid communication with a plurality of sample fluid distribution channels.

38. The measuring device of claim 36 or claim 37, comprising a plurality of the measuring systems, and wherein each of the plurality of reagent loading systems is arranged in fluid communication leading to a measuring device of the plurality of measuring systems.

39. The measuring device of claim 38 when dependent upon claim 35, comprising a plurality of the mixers, and wherein each of the plurality of reagent pre-loading systems is arranged in fluid communication with a mixer of the plurality of mixers.

40. The measuring device of any one of claims 34 to 39, wherein the sample fluid is blood.

41. The measuring device of any one of claims 34 to 40 wherein the measuring system is a point of care diagnostic measuring system for measuring one or more properties of the sample fluid either alone or mixed with a chemical or reagent.

42. The measuring device of any one of claims 34 to 41, wherein the measuring device is configured for measuring a rheology of the sample fluid.

43. The measuring device of any one of claims 34 to 42, comprising the reagent-pre-loading system of any one of claims 1 to 32.

44. A method of operating a reagent pre-loading system of a measuring device, the reagent pre-loading system comprising:
a reagent admission arrangement arranged for receiving a reagent into the reagent pre-loading system;
a sample admission arrangement arranged for receiving a sample fluid to be measured into the reagent pre-loading system,
wherein the reagent admission arrangement and the sample admission arrangement are each arranged in selective fluid communication with the sample and reagent combination conduit;
the method comprising:
whilst the sample and reagent combination conduit is closed to fluid communication with the sample fluid at the sample admission arrangement, pre-loading a reagent into the sample and reagent combination conduit at any time prior to the admission of the sample fluid into the reagent pre-loading system, so as to remove air located in the sample and reagent combination conduit as the reagent is received therein and prime the sample and reagent combination conduit with the reagent; whilst the sample and reagent combination conduit is closed to fluid communication with sample fluid at the sample admission arrangement, admitting a sample fluid into the sample admission arrangement so as to expel air from the sample admission arrangement and prime the sample admission arrangement with the sample fluid; and, whilst the sample and reagent combination conduit is open to fluid communication with the sample admission arrangement, admitting further sample fluid into the sample admission arrangement and into the sample and reagent combination conduit for combining with the reagent.

45. The method of claim 44, wherein the air located in the sample and reagent combination conduit is removed to the atmosphere via an air vent or valve.

46. The method of claim 44 or claim 45 wherein the reagent admission arrangement comprises a reagent valve arrangement, configured for selective admission of the reagent into the reagent pre-loading system, and the sample admission arrangement comprises a sample valve arrangement configured for selective admission of the sample fluid into the reagent pre-loading system.

46. The method of claim 46 when dependent on claim 45, wherein the reagent valve arrangement is configured for selective fluid communication between each of a reagent delivery port, the sample and reagent combination conduit and a mixer; and
the sample valve arrangement is configured for selective fluid communication between each of a sample fluid conduit, the sample and reagent combination conduit and the air vent or valve;
wherein the admitting the reagent into the sample and reagent combination conduit and removing air from the sample and reagent combination conduit comprises: selectively admitting the reagent at the reagent delivery port into the sample and reagent combination conduit whilst the sample valve arrangement is closed to the sample fluid conduit and open to the air vent or valve, such that admission of the reagent into the sample and reagent combination conduit displaces air in the sample and reagent combination conduit through the air vent or valve.

47. The method of claim 46, wherein the admitting the fluid sample so as to expel air from the sample valve arrangement and prime the sample valve arrangement with the sample fluid comprises: closing the sample valve arrangement to the sample and reagent combination conduit and opening the sample valve arrangement to the sample fluid conduit so as to selectively admit sample fluid into the sample fluid conduit, such that admission of the sample fluid into the sample fluid conduit displaces air in the sample fluid conduit and the sample valve arrangement through the air vent.

48. The method of claim 47, wherein admitting further fluid sample into the sample valve arrangement and into the sample and reagent combination conduit for combining with the reagent comprises, whilst the reagent valve arrangement is open to the mixer conduit and closed to the reagent delivery port: closing the sample valve arrangement to the air vent or valve and opening the sample valve arrangement to the sample and reagent combination conduit, such that further sample fluid delivered into the sample fluid conduit is admitted into the sample and reagent combination conduit for combining with the reagent in the sample and reagent combination conduit.

49. The method of claim 45, wherein the reagent admission arrangement comprises a reagent delivery port, and a pierceable sealed membrane arranged to sealingly close the reagent delivery port to fluid flow there through until it is pierced, and pre-loading the reagent into the sample and reagent combination conduit comprises piercing the sealed membrane with an external actuator or device and providing the reagent into the reagent pre-loading system via the pierced membrane.

50. A method of measuring a property of a sample fluid or other material using the measuring device of any one of claims 34 to 43, comprising:
pre-loading a reagent into the reagent loading system of the measuring device at any time prior to admission of the sample fluid into the measuring device so as to remove air located in the sample and reagent combination conduit and prime the sample and reagent combination conduit with the reagent; admitting a sample fluid into the measuring device so as to expel air from the sample admission arrangement and prime the sample admission arrangement with the sample fluid; and admitting further sample fluid into the sample admission arrangement and into the sample and reagent combination conduit for combining with the reagent; mixing the sample fluid and the reagent and measuring one or more properties of the sample fluid in the measuring system.

51. The steps, features, integers, compositions and/or compounds disclosed herein or indicated in the specification of this application individually or collectively, and any and all combinations of two or more of said steps or features.
